(19) United States
(12) Patent Application Publication
(10) Pub. No.: US 2026/0194506 A1
(43) Pub. Date: Jul. 9, 2026

(54) ELECTROCHEMICAL NANO-SENSOR ARRAY/WORKING ELECTRODES FOR METAL DETECTION, METHODS OF PRODUCING SAME AND RELATED REAGENTS

(71) Applicants: SAMI SHAMOON COLLEGE OF ENGINEERING, Beer Sheva (IL); NUCLEAR RESEARCH CENTER, Beer Sheva (IL)

(72) Inventors: Ariela Burg, Beer Sheva (IL); Dror SHAMIR, Gvaot Bar (IL); Moshe ZOHAR, Beer Sheva (IL)

(73) Assignees: SAMI SHAMOON COLLEGE OF ENGINEERING, Beer Sheva (IL); NUCLEAR RESEARCH CENTER, Beer Sheva (IL)

(21) Appl. No.: 18/867,849

(22) PCT Filed: May 17, 2023
(86) PCT No.: PCT/IL2023/050511

§ 371 Date: Nov. 21, 2024

Related U.S. Application Data
(60) Provisional application No. 63/345,050, filed on May 24, 2022.

(51) Int. Cl. G01N 33/18; G01N 27/30

(52) U.S. Cl. CPC G01N 33/1813; G01N 27/30

(57) ABSTRACT

A working electrode comprising: (a) a conductive surface; and (b) a plurality of nanoclusters of one or more ligands specific to one or more metals distributed on the conductive surface with a controlled pitch. Related methods and compositions are also disclosed.

### RELATED APPLICATIONS

This PCT application claims the benefit according to 35 U.S.C. § 119 (e) of U.S. provisional patent application 63/345,050 filed on May 24, 2022 and having the same title and applicant as the present application which is fully incorporated herein by reference and publicly available subsequent to publication of the present application.

### FIELD OF THE INVENTION

The invention is in the field of electrochemical detection.

### BACKGROUND OF THE INVENTION

Rapid global population growth has led to rampant industrial development around the world and the consequent pollution of water sources with heavy metals and toxic organic waste. Non-biodegradable heavy metals are a major source of environmental pollution. These metals are highly toxic, and carcinogenic even at trace levels, are ubiquitous in the environment and are often found in treated waste waters thus endangering global sustainability.

Therefore, water purification treatments should be followed by the use of efficient and sensitive methods to detect the presence of metal ions, such as cadmium, chromium, lead, mercury, copper, nickel, vanadium, molybdenum, antimony and uranium in the treated water. These heavy metal ions are hazardous pollutants, ranking in the top ten on the Priority List of Hazardous Substances of the Agency for Toxic Substances and Disease Registry.

Heavy metal concentrations in drinking water must abide by local safety standards, and in most locations and for most metals, regulations typically allow concentrations of no more than a few parts per billion.

Countless studies have therefore been done to develop heavy metal sensors with appropriate sensitivity. The detection of trace amounts of heavy metals is typically performed with traditional analytical techniques that rely on various instruments and a range of methods such as atomic absorption spectrometry, inductively coupled plasma, optical techniques and Raman scattering spectroscopy.

However, most of those techniques require expensive instruments and/or extensive sample preparation and/or specialized personnel and/or long training times for personnel to perform the analysis. In addition, the pre-analysis preparation of the sample is typically not done on-site. The subsequent transportation, handling, and pre-treatment of the samples prior to their analysis favors the occurrence of undesirable chemical, biological and physical reactions that can alter the sample's composition, thus increasing the potential of obtaining inaccurate results.

### SUMMARY OF THE INVENTION

A broad aspect of the invention relates to electrochemically-based sensors for in situ analysis of heavy metals. Electrochemical sensors have significant advantages relative to other detection methods. These advantages include but are not limited to portability and ease of sample preparation. In some embodiments, portability contributes to an ability to perform measurements in the field.

Electrochemical detection has to give a specific electrical signal for each cation in a solution that includes one or more cations. Interference (e.g. matrix interference) and/or masking of the signal by other cations or their signals in the solution (i.e., interfering with signal intensity and/or current and/or the redox potential) is a problem in many existing electrochemical sensor arrays. Electrochemical measurements can be either direct or indirect. Indirect sensors are based on inhibiting enzymes that are used to quantify the amount of cation by measuring the level of enzyme inhibition. Direct sensors rely on measurements using a ligand with specificity to one or more heavy metal ions. Many exemplary embodiments of the invention rely on direct measurements.

One aspect of some embodiments of the invention relates to a working electrode comprising a plurality of nanoclusters made from ink deposited at defined locations. For purposes of this specification and the accompanying claims, the term “ink” indicates a mixture including a solvent (inert with respect to the detection process) and a ligand specific to one or more heavy metals. In some exemplary embodiments of the invention, a liquid ligand is applied directly as an ink without solvent. In some exemplary embodiments of the invention, specificity of an ink for a specific heavy metal is determined by the ligand. Alternatively or additionally, in some embodiments ligand concentration and/or solvent contribute to detection sensitivity. In some embodiments a patterning method (such as dip pen nanolithography (DPN)) is used to produce patterns of different inks on a same working electrode. According to various exemplary embodiments of the invention the patterns are separate and/or parallel and/or overlapping. In some embodiments a size of the nanocluster is controlled during ink deposition. Alternatively or additionally, in some embodiments nanoclusters are distributed on a conductive surface with a controlled pitch. For purposes of this specification and the accompanying claims, the term “pitch” (P) refers to a distance between the center of adjacent nanoclusters. For purposes of this specification and the accompanying claims, the term “diameter” refers to the maximum distance between two opposite sides or edges of the base of the cluster. In some embodiments pitch (P) is controlled separately on the X-Axis (Pₓ) and on the Y axis (P_{y}) In some exemplary embodiments of the invention, an ability to control nanocluster size and pitch between nanoclusters contributes to the current and/or the redox potential. In some embodiments control of the current and/or the redox potential contributes to a decrease in interference caused by other heavy metals in the sample. Alternatively or additionally, in some embodiments different nanocluster types (i.e. different ligands) patterned on the same surface detect different heavy metals in parallel in a single sensor array. In some exemplary embodiments of the invention, during ink deposition on the reactive surface, nanoclusters with different ligands are patterned on the reactive surface. According to these embodiments, the different ligands detect different heavy metals in response to a user input signal. Alternatively or additionally, in some embodiments the pitch is adjusted to decrease interference and/or to increase detection sensitivity.

In some exemplary embodiments of the invention, control of nanocluster size, especially the surface area to volume ratio of the cluster, and/or the pattern pitch contributes, to an increase in sensitivity and/or an increase in specificity. (See in this regard Table 3, Table 4, and FIGS. 11, 12, 13, 14, 15, 16, 17, 18a, and 18b) In some embodiments current is an indicator of sensitivity. For example, if the current is higher, sensitivity is higher. According to various exemplary embodiments of the invention, the conductive surface includes ITO (indium tin oxide) and/or FTO (fluorine doped tin oxide) and/or Pt (platinum) and/or Au (gold) and/or Glassy carbon and/or semiconductor materials. Alternatively or additionally, in various embodiments the solvent includes water and/or Acetonitrile and/or conductive polymers such as poly(3,4-ethylenedioxythiophene):poly(styrene sulfonate) (PEDOT:PSS) and/or Nafion (sulfonated tetrafluoroethylene-based fluoropolymer-copolymer) and/or silane compounds such as TMOS (Tetramethoxysilane) and/or TEOS (Tetraethoxysilane) and/or TMOP (Trimethoxyphenylsilane) and/or polymers in liquid formulations (PLFs) include different polymer types such as acrylic, epoxy resins, polyesters, polysilicon, polyurethanes, radiation curable, vinyl, water-soluble polymers such as PDMS (Polydimethylsiloxane) and/or PMMA (Polymethyl methacrylate). Alternatively or additionally, in various embodiments the ligand includes phosphonate compounds and/or carbonate compounds and/or amine compounds and/or thiol compounds and/or crown ether compounds (for example) penicillamine (PNA) and/or nitrilo triacetic acid (NTA) and/or dipicolinic acid (DPA), and/or nitrilotris(methylene)] tris (phosphonic acid (ATMP) and/or 1,8-diamininaphtalene (DAN) and/or N-hydroxysuccinimide and/or riboflavin-5-phosphate and/or cupferron and/or tributyl phosphate and/or glutathione and/or black phosphorus and/or graphene and/or mercury and/or bismuth.

Another aspect of some embodiments of the invention relates to the application of a solution or suspension of ligand dissolved/suspended in a solvent to a conductive surface. In some embodiments, patterning lithography is used for deposition. In some embodiments using dip pen nanolithography (DPN) is used for deposition. One example of a DPN device suitable for use in the context of these embodiments of the invention is the NLP 2000 (NANOINK INC. nano fabrication systems; USA). A person of ordinary skill in the art will be capable of selecting other DPN devices or/and other lithography devices such as soft lithography and/or nanoimprint lithography devices with similar capabilities and/or of scaling up the process by using a device with a larger capacity. During application, the size of the nanoclusters being applied and their pitch is controlled to achieve a desired sensitivity and/or specificity. The ability to control the pitch and to control the size of the nanoclusters contributes to control of electrical signals and the sensitivity of the detection and/or to a decrease in interference from other cations in the solution. Alternatively or additionally, in some embodiments, the concentration of ligand dissolved/suspended in the solvent contributes to sensitivity and/or specificity. The conductive surfaces, ligands and solvents are as described in the context of the previous aspect.

Another aspect of some embodiments of the invention relates to patterning lithography ink comprising a ligand with specificity to one or more heavy metals dissolved/suspended in a solvent or mixture of solvents. The ligands and solvents are as described in the context of the previous aspects. According to various exemplary embodiments of the invention, ink includes at least 1%, at least 5%, at least 10%, at least 15%, at least 20%, at least 30%, at least 40%, at least 50%, at least 60%, at least 70%, at least 75%, at least 80%, at least 85%, at least 90%, at least 95%, at least 97.5%, at least 99% or intermediate or greater percentages of solvent by volume. Alternatively or additionally, according to various exemplary embodiments of the invention the ink includes less than 99%, less than 97.5%, less than 95%, less than 90%, less than 85%, less than 80%, less than 75%, less than 70%, less than 60%, less than 50%, less than 40%, less than 30%, less than 20%, less than 15%, less than 10%, less than 5%, less than 1% or intermediate or lower percentages of solvent by volume. In some exemplary embodiments of the invention, the solvent includes PMMA, In other exemplary embodiments of the invention, other polymers are employed.

While the aspects are presented separately above, two or more aspects, or their features, are often combined to produce additional embodiments of the specification.

It will be appreciated that the various aspects described above relate to the solution of technical problems associated with increasing sensitivity of detection of heavy metals using a direct electrochemical sensor.

Alternatively or additionally, it will be appreciated that the various aspects described above relate to solution of technical problems related to improving portability of heavy metal detection devices.

Alternatively or additionally, it will be appreciated that the various aspects described above relate to solution of technical problems related to measuring two or more heavy metals in a sample using a single working electrode.

In some exemplary embodiments of the invention there is provided a working electrode including: (a) a conductive surface; and (b) a plurality of nanoclusters of one or more ligands specific to one or more heavy metals distributed on the conductive surface with a controlled pitch. In some embodiments the nanoclusters have a height of at least 2 nm, at least 3 nm, at least 4 nm, at least 5 nm or intermediate or greater heights. Alternatively or additionally, in some embodiments the nanoclusters have a height of less than 6 nm, less than 5 nm, less than 4 nm, less than 3 nm or intermediate or smaller heights. Alternatively or additionally, in some embodiments the nanoclusters have a height of 1000 nm or less. Alternatively or additionally, in some embodiments the nanoclusters have a diameter of at least 10 nm, at least 20 nm, at least 30 nm, at least 40 nm, at least 50 nm or intermediate or greater diameters. Alternatively or additionally, in some embodiments the nanoclusters have a diameter of less than 50 nm, less than 40 nm, less than 30 nm, less than 20 nm, or intermediate or smaller diameters. Alternatively or additionally, in some embodiments the nanoclusters have a diameter of 6000 nm or less. Alternatively or additionally, in some embodiments the controlled pitch is at least 10 nm, at least 20 nm, at least 30 nm or intermediate or greater numbers of nm. Alternatively or additionally, in some embodiments the controlled pitch is 11,000 nm or less, 10,000 nm or less, 1000 nm or less, 900 nm or less, 800 nm or less, 700 nm or less, 600 nm or less, 500 nm or less, 400 nm or less, 300 nm or less, 200 nm or less, 100 nm or less or intermediate or lower values. Alternatively or additionally, in some embodiments the conductive surface includes at least one member of the group consisting of ITO (indium tin oxide), FTO (fluorine doped tin oxide), Pt (platinum), Au (gold), Glassy carbon and semiconductor materials. Alternatively or additionally, in some embodiments the nanoclusters includes one or more residual solvents selected from the group consisting of Water, Acetonitrile, conductive polymers such as poly(3,4-ethylenedioxythiophene):poly(styrene sulfonate) (PEDOT:PSS), Nafion (sulfonated tetrafluoroethylene-based fluoropolymer-copolymer), silane compounds such as TMOS (Tetramethoxysilane), TEOS (Tetraethoxysilane), TMOP (Trimethoxyphenylsilane), polymers in liquid formulations (PLFs) (e.g. acrylic, epoxy resins, polyesters, polysilicon, polyurethanes, radiation curable, vinyl, water-soluble polymers such as PDMS (Polydimethylsiloxane) and PMMA (Polymethyl methacrylate)). Alternatively or additionally, in some embodiments the nanoclusters include one or more ligands selected from the group consisting of phosphonate compounds, carbonate compounds, amine compounds, thiol compounds, crown ether compounds (e.g. penicillamine (PNA)), nitrilo triacetic acid (NTA), dipicolinic acid (DPA), nitrilotris(methylene)] tris (phosphonic acid (ATMP), and Glutathione. Alternatively or additionally, in some embodiments the electrode is configured to detect at least two metals selected from the group consisting of cadmium, chromium, lead, mercury, nickel, copper, cerium, vanadium, molybdenum, vanadium, molybdenum, and uranium (in their various oxidation states) concurrently in a single sample. Alternatively or additionally, in some exemplary embodiments of the invention, the electrode is configured to detect at least two metals concurrently in a single sample. In some embodiments the at least two metals are selected from the group consisting of cadmium, chromium, lead, mercury, nickel, copper, cerium, vanadium, molybdenum, and uranium (in their various oxidation states).

In some exemplary embodiments of the invention there is provided a method including: applying one or more solutions or suspensions of ligand(s) specific to one or more heavy metals dissolved/suspended in one or more solvents to a conductive surface using patterning lithography to produce nanoclusters with a controlled pitch. In some embodiments the method includes controlling the size of the nanoclusters. Alternatively or additionally, in some embodiments the controlling includes ensuring the nanoclusters have a height of 3 nm to 1000 nm. Alternatively or additionally, in some embodiments the controlling includes ensuring the nanoclusters have a diameter 10 nm to 6000 nm. In some embodiments the diameter is 30 nm or more. Alternatively or additionally, in some embodiments the controlled pitch is 10 nm to 11,000 nm. In some embodiments the controlled pitch is 30 nm or more. Alternatively or additionally, in some embodiments the method includes dissolving the ligand(s) in the one or more solvents at a concentration of 1.00×10⁻⁸ M to 0.75 M. Alternatively or additionally, in some embodiments the conductive surface includes at least one member of the group consisting of ITO (indium tin oxide), FTO (fluorine doped tin oxide), Pt (platinum), Au (gold), Glassy carbon, and semiconductor materials. Alternatively or additionally, in some embodiments the one or more solvents include at least one member of the group consisting of Water, Acetonitrile, conductive polymers such as poly(3,4-ethylenedioxythiophene):poly(styrene sulfonate) (PEDOT:PSS), Nafion (sulfonated tetrafluoroethylene-based fluoropolymer-copolymer), silane compounds such as TMOS (Tetramethoxysilane), TEOS (Tetraethoxysilane), TMOP (Trimethoxyphenylsilane), polymers in liquid formulations (PLFs) such as acrylic, epoxy resins, polyesters, polysilicon, polyurethanes, radiation curable, vinyl, water-soluble polymers. Alternatively or additionally, in some embodiments the ligand(s) specific to one or more heavy metals include at least one member selected from the group consisting of phosphonate compounds, carbonate compounds, amine compounds, thiol compounds, crown ether compounds (e.g. penicillamine (PNA)), nitrilo triacetic acid (NTA), dipicolinic acid (DPA), nitrilotris(methylene)] tris (phosphonic acid (ATMP), and Glutathione.

In some exemplary embodiments of the invention there is provided a patterning ink including: a ligand with specificity to one or more metals (e.g. heavy metals) dissolved/suspended in a solvent or mixture of solvents. In some embodiments the ink includes at least 1% solvent by volume. Alternatively or additionally, in some embodiments the ink includes less than 75% solvent by volume. Alternatively or additionally, in some embodiments the ink includes from 1.00×10⁻⁸ M to 0.75 M of the ligand. Alternatively or additionally, in some embodiments the solvent or mixture of solvents includes at least one member of the group consisting conductive of Water, Acetonitrile, polymers such as poly(3,4-ethylenedioxythiophene):poly(styrene sulfonate) (PEDOT:PSS), Nafion (sulfonated tetrafluoroethylene-based fluoropolymer-copolymer), silane compounds such as TMOS (Tetramethoxysilane), TEOS (Tetraethoxysilane), TMOP (Trimethoxyphenylsilane), polymers in liquid formulations (PLFs) such as acrylic, epoxy resins, polyesters, polysilicon, polyurethanes, radiation curable, vinyl, water-soluble, PDMS (Polydimethylsiloxane), and PMMA (Polymethyl methacrylate). Alternatively or additionally, in some embodiments the ligand includes at least one member selected from the group consisting of phosphonate compounds, carbonate compounds, amine compounds, thiol compounds, crown ether compounds (for example) penicillamine (PNA), nitrilo triacetic acid (NTA), dipicolinic acid (DPA), nitrilotris(methylene)] tris (phosphonic acid (ATMP), 1,8-diamininaphtalene (DAN), N-hydroxysuccinimide, riboflavin-5-phosphate, cupferron, r tributyl phosphate, glutathione, black phosphorus, graphene and/or mercury, and bismuth.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system according to embodiments of the invention involves performing or completing selected tasks or steps manually, automatically, or a combination thereof. Moreover, according to actual instrumentation and equipment of exemplary embodiments of methods, apparatus and systems of the invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Percentages (%) are V/V (volume to volume) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a simplified schematic representation of a working electrode according to some exemplary embodiments of the invention;

FIG. 2 is a histogram of current [μA] as a function of Potential vs. Au/V for MICRUX™ electrode AuAuAuPMMANTPH6.6 (E deposition=−1.1 (V); t deposition=120 s) during linear sweep voltammetry in a solution including 10 ppb UO₂(NO₃)₂, 1.0×10⁻⁶ M KNO₃, pH 4.0;

FIG. 3 is a schematic representation of a Thin-film Gold MICRUX™ (Micrux Technologies; Spain) single electrode (AuAuAu) suitable for use in some embodiments of the invention;

FIG. 4 is a heat map of thickness produced by Matlab analysis of AuAuAuPMMANTPH pattern scanning;

FIG. 5 is a histogram of current [μA] as a function of Potential vs. Au/V for MICRUX™ electrode AuAuAuPMMANTPH6.6 (E deposition=−1.1 (V); t deposition=60 s; Frequency=25 Hz; Amplitude=0.1V) during square wave anodic stripping in a solution including Pb(NO₂)₂, 1.0×10⁻⁶ M KNO₃, pH 5.5;

FIG. 6 is a histogram of current [μA] as a function of Potential vs. Au/V for MICRUX™ electrode AuAuAuPMMAGlu6.6 (E deposition=−1.1 (V); t deposition=60 s; Frequency=25 Hz; Amplitude=0.1V) during square wave anodic stripping in a solution including Pb(NO₂)₂ and Hg(NO₂)₂, 1.0×10⁻⁶ M KNO₃, pH 5.5;

FIG. 7 is a histogram of current [μA] as a function of Potential vs. Au/V for MICRUX™ electrode AuAuAuPMMADPA6.6 (E deposition=−1.1 (V); t deposition=60 s; Frequency=25 Hz; Amplitude=0.075V) during square wave anodic stripping in a solution including Pb(NO₂)₂ and Cu(NO₂)₂, ionic strength 5.0×10⁻⁵ M, pH 3.0;

FIG. 8 is a histogram of current [μA] as a function of Potential vs. Au/V for MICRUX™ electrode AuAuAuPMMADAN6.6 (E deposition=−1.2 (V); t deposition=60 s; Frequency=25 Hz; Amplitude=0.01V) during square wave anodic stripping in a solution including Pb(NO₂)₂ and Cd(NO₂)₂, ionic strength 5.0×10⁻⁵M, pH 3.0;

FIG. 9 is a histogram of current [μA] as a function of Potential vs. Au/V for MICRUX™ electrode AuAuAuPMMAHg6.6 (E deposition=−1.2 (V); t deposition=60 s; Frequency=25 Hz; Amplitude=0, 1V) during square wave anodic stripping in a solution including Cd(NO₂)₂ Pb(NO₂)₂ and Cu(NO₂)₂, ionic strength 5.0×10⁻⁵ M, buffer acetate pH 4.6. Gray line; The solutions include ionic strength 5.0×10⁻⁵ M, buffer acetate pH 4.6;

FIG. 10 is a histogram of current [μA] as a function of Potential vs. Au/V for MICRUX™ electrodes (E deposition=−1.2 (V); t deposition=60 s; Frequency=25 Hz; Amplitude=0, 1V). The solutions include ionic strength 5.0×10⁻⁵ M, Buffer acetate pH 4.6. Gray line—AuAuAuPMMAHg6.6 electrode, 0.10 ppb Cd(NO₂)₂ Pb(NO₂)₂ and Cu(NO₂)₂, [Black line—AuAuAu electrode, Buffer acetate pH 4.6. Broken black line-AuAuAu electrode, 1.0 ppb Cd(NO₂)₂ Pb(NO₂)₂ and Cu(NO₂)₂, Dotted black line AuAuAuPMMA electrode, 1.0 ppb Cd(NO₂)₂ Pb(NO₂)₂ and Cu(NO₂)₂];

FIG. 11 is a histogram of current [nA] as a function of [Hg(NO₂)₂] serving as a calibration curve for MICRUX™ electrode AuAuAuPMMAGlu (E deposition=−1.1 (V); t deposition=60 s; Frequency=25 Hz; Amplitude=0.1V) in a solution including Buffer acetate pH 4.6. Ionic strength 5.0×10⁻⁵ M;

FIG. 12 is a histogram of current [μA] as a function of Potentialvs. Au/V for MICRUX™ electrode AuAuAuPMMAGlu (E deposition=−1.1 (V); t deposition=60 s; Frequency=25 Hz; Amplitude=0.1V) during square wave anodic stripping in a solution including 0.50 ppb Hg(NO₂)₂, Buffer acetate pH 4.6. Ionic strength 5.0×10⁻⁵ M;

FIG. 13 is a histogram of current [μA] as a function of Potentialvs. Au/V for MICRUX™ electrode AuAuAuPMMAGlu (E deposition=−1.1 (V); t deposition=60 s; Frequency=25 Hz; Amplitude=0.1V) during square wave anodic stripping in a solution including 1.0 ppb Hg(NO₂)₂, Buffer acetate pH 4.6. Ionic strength 5.0×10⁻⁵ M;

FIG. 14 is a histogram of current [μA] as a function of Potential vs. Au/V for MICRUX™ electrode AuAuAuPMMAGlu (E deposition=−1.1 (V); t deposition=60 s; Frequency=25 Hz; Amplitude=0.1V) during square wave anodic stripping in a solution including 2.0 ppb Hg(NO₂)₂, Buffer acetate pH 4.6. ionic strength 5.0×10⁻⁵ M;

FIG. 15 is a histogram of current [μA] as a function of Potential vs. Au/V for MICRUX™ electrode AuAuAuPMMAGlu (E deposition=−1.1 (V); t deposition=60 s; Frequency=25 Hz; Amplitude=0.1V) during square wave anodic stripping in a solution including 4.0 ppb Hg(NO₂)₂, Buffer acetate pH 4.6. Ionic strength 5.0×10⁻⁵ M;

FIG. 16 is a histogram of current [μA] as a function of Potential vs. Au/V for MICRUX™ electrode AuAuAuPMMAGlu (E deposition=−1.1 (V); t deposition=60 s; Frequency=25 Hz; Amplitude=0.1V) during square wave anodic stripping in a solution including 0.50 ppb Hg(NO₂) 2 and 0.50 ppb Pb(NO₂)₂, Buffer acetate pH 4.6. Ionic strength 5.0×10⁻⁵ M;

FIG. 17 is a histogram of current [μA] as a function of [Pb(NO₂)₂] serving as a calibration curve for MICRUX™ electrodes AuAuAuPMMANTPH6.6 (E deposition=−1.1 (V); t deposition=60 s; Frequency=25 Hz; Amplitude=0.1V) in a solution including buffer acetate pH 4.6; ionic strength 5.0×10⁻⁵ M, Pb(NO₂)₂;

FIG. 18a is a calibration curve of current [μA] as a function of [Hg(NO₂)₂] of MICRUX™ electrode AuAuAuPMMAGlu6.6 (E deposition=−1.1 (V); t deposition=60 s; Frequency=25 Hz; Amplitude=0.1V) in a solution including buffer acetate pH 4.6. Ionic strength 5.0×10⁻⁵ M Hg(NO₂)₂; and

FIG. 18b is a calibration curve of current [μA] as a function of [Pb(NO₂)₂] of MICRUX™ electrode AuAuAuPMMAGlu6.6 (E deposition=−1.1 (V); t deposition=60 s; Frequency=25 Hz; Amplitude=0.1V). in a solution including buffer acetate pH 4.6. Ionic strength 5.0×10⁻⁵ M Pb(NO₂)₂.

### DETAILED DESCRIPTION OF EMBODIMENTS

Embodiments of the invention relate to working electrodes, sensor arrays comprising them, methods of producing the working electrodes, and inks used in that production.

Specifically, some embodiments of the invention can be used to provide direct electrochemical detection of one or more metal cations in a solution. In some exemplary embodiments of the invention, the metals are heavy metals.

The principles and operation of a working electrode, production method and ink according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

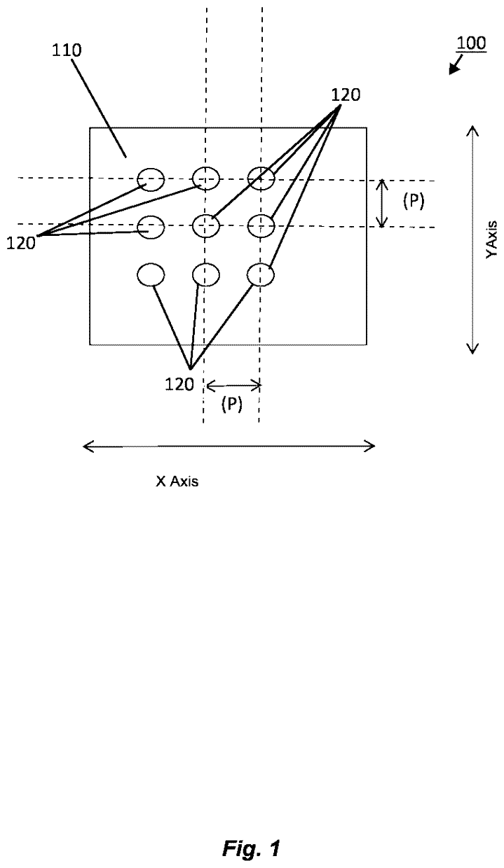

FIG. 1 is a simplified schematic representation of a working electrode for direct electrochemical measurement of metal ion concentration in solution, generally indicated as 100, according to some exemplary embodiments of the invention. Depicted exemplary electrode 100 includes a conductive surface 110 with a plurality of nanoclusters 120 distributed in a regular pattern on its surface. Each nanocluster 120 includes one or more ligands specific to one or more heavy metals. There is a controlled pitch (P) between the center of adjacent nanoclusters 120.

In some exemplary embodiments of the invention, nanoclusters 120 have a height of at least 3 nm, at least 5 nm, at least 10 nm, at least 20 nm, at least 50 nm, at least 100 nm, at least 100 nm, at least 200 nm, at least 300 nm, at least 400 nm, at least 500 nm, at least 600 nm, at least 700 nm, at least 800 nm, at least 900 nm, at least 1000 nm or intermediate or greater heights. Alternatively or additionally, in some embodiments nanoclusters 120 have a height of 1000 nm. 900 nm, 900 nm, 800 nm, 700 nm, 600 nm, 500 nm, 400 nm, 300 nm, 200 nm, 100 nm, 50 nm, 20 nm, 10 nm, 5 nm, 3 nm, or intermediate or lesser heights.

Alternatively or additionally, in some embodiments nanoclusters 120 have a diameter of at least 10 nm, at least 20 nm, at least 30 nm, at least 50 nm, at least 100 nm, at least 250 nm, at least 500 nm, at least 1000 nm, at least 2000 nm, at least 3000, nm, at least 4000 nm at least 5000 nm, at least 6000 nm or intermediate or greater diameters. Alternatively or additionally, in some embodiments nanoclusters 120 have a diameter of 6000 nm or less, 5000 nm or less, 4000 nm or less, 3000 nm or less, 2000 nm or less, 1000 nm or less, 500 nm or less, 250 nm or less, 100 nm or less, 75 nm or less, 50 nm or less, 30 nm or less, 20 nm or less, 10 nm or less, or intermediate or smaller diameters.

Alternatively or additionally, in some embodiments controlled pitch (P) is at least 10 nm, at least 20 nm, at least 30 nm, at least 50 nm, at least 100 nm, at least 500 nm, at least 1000 nm, at least 2000 nm, at least 3000 nm, at least 4000 nm, at least 5000 nm, at least 6000 nm, at least 7000 nm, at least 8000 nm, at least 9000 nm, at least 10,000 nm, at least 11,000 nm, or intermediate or greater distances. Alternatively or additionally, in some embodiments controlled pitch (P) is 11,000 nm or less, 10,000 nm or less, 9000 nm or less, 8000 nm or less, 7000 nm or less, 6000 nm or less, 5000 nm or less, 2000 nm or less, 1000 nm or less, 500 nm or less, 100 nm or less, 50 nm or less, 30 nm or less, 20 nm or less, 10 nm or less, or intermediate or smaller distances.

Alternatively or additionally, in some embodiments conductive surface 110 includes ITO (indium tin oxide) and/or FTO (fluorine doped tin oxide) and/or Pt (platinum) and/or Au (gold), and/or Glassy carbon and/or semiconductor materials.

Alternatively or additionally, in some embodiments nanoclusters 120 include one or more residual solvents. Exemplary solvents include but are not limited to Water and/or

Acetonitrile and/or conductive polymers such as poly(3,4-ethylenedioxythiophene):poly(styrene sulfonate) (PEDOT:PSS) and/or Nafion (sulfonated tetrafluoroethylene-based fluoropolymer-copolymer) and/or silane compounds such as TMOS (Tetramethoxysilane) and/or TEOS (Tetraethoxysilane) and/or TMOP (Trimethoxyphenylsilane) and/or polymers in liquid formulations (PLFs) such as acrylic, epoxy resins, polyesters, polysilicon, polyurethanes, radiation curable, vinyl, water-soluble polymers such as PDMS (Polydimethylsiloxane) and PMMA (Polymethyl methacrylate).

Alternatively or additionally, in some embodiments nanoclusters 120 include as ligands one or more of phosphonate compounds, carbonate compounds, amine compounds, thiol compounds, crown ether compounds (e.g. penicillamine (PNA)), nitrilo triacetic acid (NTA), dipicolinic acid (DPA), nitrilotris(methylene)] tris (phosphonic acid (ATMP) and Glutathione.

Alternatively or additionally, in some embodiments working electrode 100 is configured to detect at least two metals selected from the group consisting of cadmium, chromium, lead, mercury, nickel, copper, cerium, vanadium, molybdenum and uranium (in their various oxidation states) concurrently in a single sample.

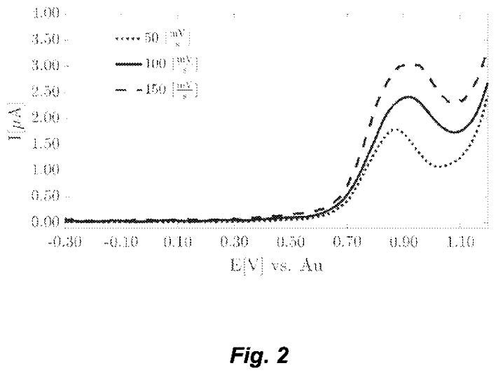

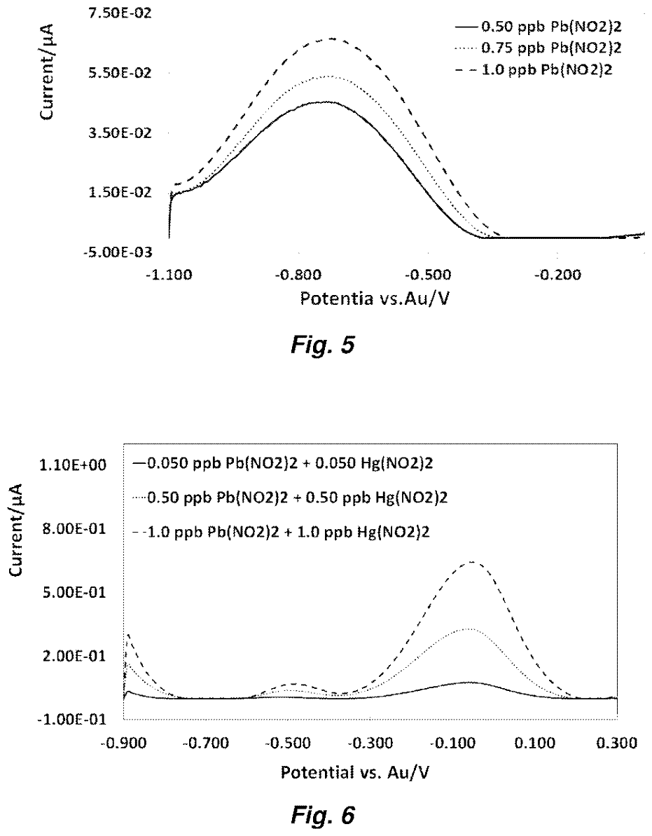

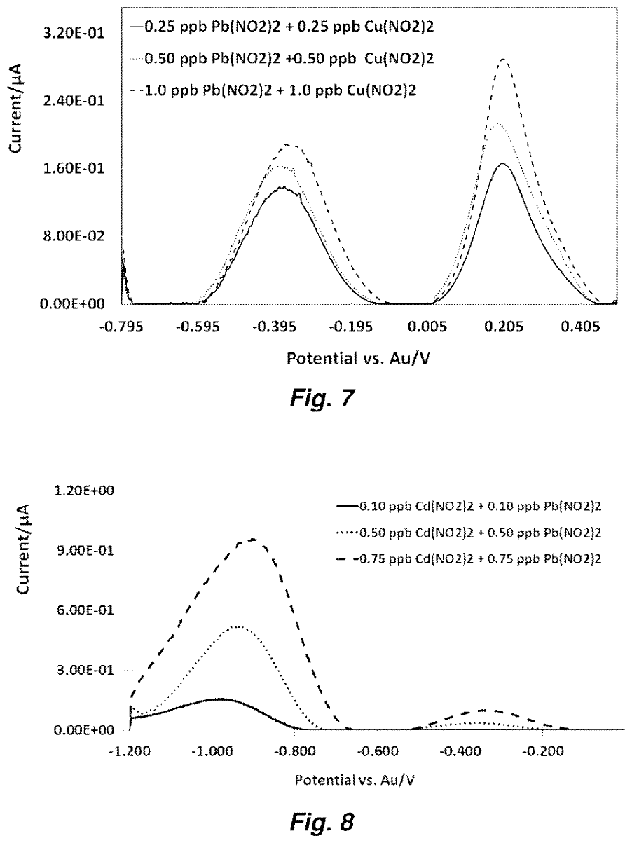

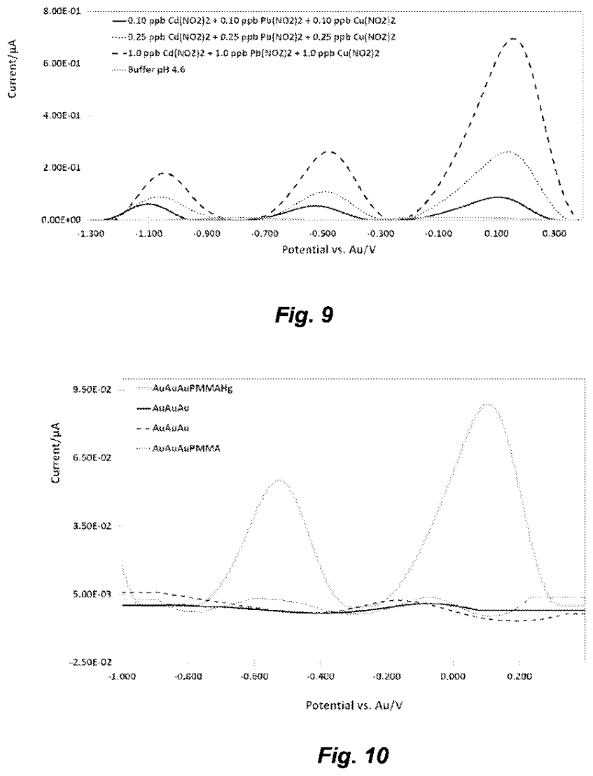

The results of FIG. 2, FIG. 5, FIG. 6, FIG. 7, FIG. 8 and FIG. 9 show that the electrical signals (the oxidation wave) of uranyl, copper, cadmium, lead and mercury, are different. The difference in electrical signals contributes to an ability to detect the two or more metals together in the same solution. FIGS. 2, and 6 through 9 demonstrate that each metal has a different signal (FIG. 2 uranyl, FIG. 6 lead and mercury; FIG. 7 lead and copper, FIG. 8 cadmium and lead, FIG. 9 cadmium, lead and copper).

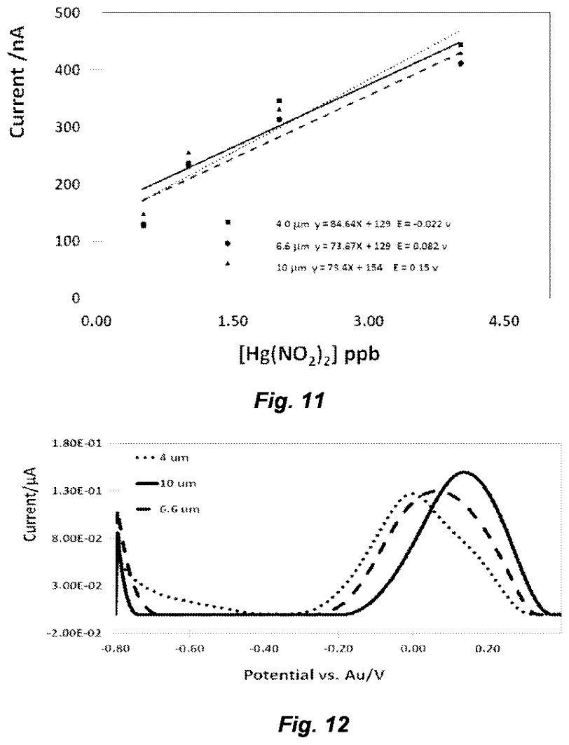

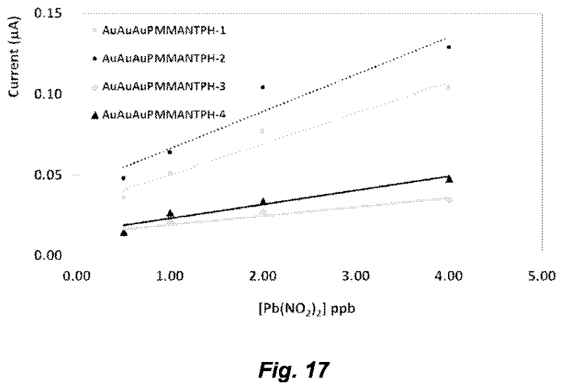

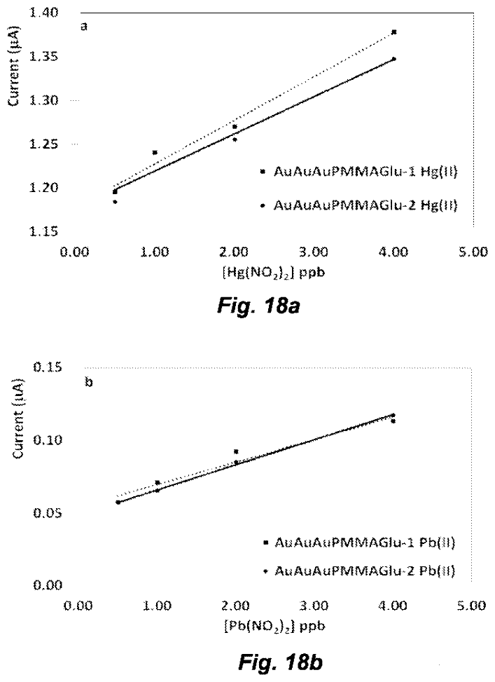

In some exemplary embodiments of the invention there is provided a method including: applying one or more solutions or suspensions of ligand(s) specific to one or more heavy metals dissolved/suspended in one or more solvents to a conductive surface using patterning lithography to produce nanoclusters with a controlled pitch (See FIGS. 11-16). In some embodiments the method includes controlling a size of the nanoclusters, (See FIGS. 17-18b and Tables 3 to 4). In some exemplary embodiments of the invention, the controlling includes insuring said nanoclusters have a height of 3 nm to 1000 nm as set forth in detail hereinabove. Alternatively or additionally, in some embodiments the controlling includes ensuring the nanoclusters have a diameter 10 nm to 6000 nm as set forth in detail hereinabove. Alternatively or additionally, in some embodiments the controlled pitch (P) is 10 nm to 11,000 nm as set forth in detail hereinabove. In some exemplary embodiments of the invention, the controlled pitch is at least 30 nm. According to various exemplary embodiments of the invention the method includes dissolving the ligand(s) in the one or more solvents at a concentration of 1.00×10⁻⁸ M, 1.00×10⁻⁷M, 1.00×10⁻⁶M, 1.00×10⁻⁵ M, 1.00×10⁻⁴ M, 1.00×10⁻³ M, 1.00×10⁻² M, 1.00×10⁻¹ M 0.75 M or intermediate or higher molar concentrations.

Alternatively or additionally, in some embodiments the method includes dissolving the ligand(s) in the one or more solvents at a concentration of 0.75 M, 1.00×10⁻¹ M, 1.00×10⁻² M, 1.00×10⁻³ M, 1.00×10⁻⁴ M, 1.00×10⁻⁵ M, 1.00×10⁻⁶M, 1.00×10⁻⁷M, 1.00×10⁻⁸ M or intermediate or lower molar concentrations.

In some exemplary embodiments of the method, the conductive surface includes ITO (indium tin oxide) and/or FTO (fluorine doped tin oxide) and/or Pt (platinum) and/or Au (gold) and/or Glassy carbon and/or semiconductor material(s).

Alternatively or additionally, in some embodiments of the method the one or more solvents include Water and/or Acetonitrile and/or conductive polymers such as poly(3,4-ethylenedioxythiophene):poly(styrene sulfonate) (PEDOT:PSS) and/or Nafion (sulfonated tetrafluoroethylene-based fluoropolymer-copolymer) and/or silane compounds such as TMOS (Tetramethoxysilane) and/or TEOS (Tetraethoxysilane) and/or TMOP (Trimethoxyphenylsilane) and/or polymers in liquid formulations (PLFs) such as acrylic, epoxy resins, polyesters, polysilicon, polyurethanes, radiation curable, vinyl, water-soluble polymers.

Alternatively or additionally, in some embodiments of the method the ligand(s) specific to one or more heavy metals, (See. FIG. 6, FIG. 7, FIG. 8 and FIG. 9) include phosphonate compounds and/or carbonate compounds and/or amine compounds and/or thiol compounds and/or crown ether compounds (e.g. penicillamine (PNA)) and/or nitrilo triacetic acid (NTA) and/or dipicolinic acid (DPA), and/or nitrilotris(methylene)] tris (phosphonic acid (ATMP) and/or 1,8-diamininaphtalene (DAN) and/or N-hydroxysuccinimide and/or riboflavin-5-phosphate and/or cupferron and/or tributyl phosphate and/or glutathione and/or black phosphorus and/or graphene and/or mercury and/or bismuth.

In some exemplary embodiments of the invention there is provided a patterning ink comprising a ligand with specificity to one or more heavy metals dissolved/suspended in a solvent or mixture of solvents. In some embodiments the ink includes at least 1% solvent by volume. Alternatively or additionally, in some embodiments the ink includes less than 75% solvent by volume. Alternatively or additionally, in some embodiments the ink includes from 1.00×10⁻⁸ M to 0.75 M of the ligand as set forth in detail hereinabove. Alternatively or additionally, in some embodiments the solvent or mixture of solvents includes Water and/or Acetonitrile and/or conductive polymers such as poly(3,4-ethylenedioxythiophene):poly(styrene sulfonate) (PEDOT:PSS) and/or Nafion (sulfonated tetrafluoroethylene-based fluoropolymer-copolymer) and/or silane compounds such as TMOS (Tetramethoxysilane) and/or TEOS (Tetraethoxysilane) and/or TMOP (Trimethoxyphenylsilane) and/or polymers in liquid formulations (PLFs) such as acrylic, epoxy resins, polyesters, polysilicon, polyurethanes, radiation curable, vinyl, water-soluble polymers such as PDMS (Polydimethylsiloxane) and PMMA (Polymethyl methacrylate). Alternatively or additionally, in some embodiments the ligand includes at least one member selected from the group consisting of phosphonate compounds and/or carbonate compounds and/or amine compounds and/or thiol compounds and/or crown ether compounds (for example) penicillamine (PNA) and/or nitrilo triacetic acid (NTA) and/or dipicolinic acid (DPA), and/or nitrilotris(methylene)] tris (phosphonic acid (ATMP) and/or 1,8-diamininaphtalene (DAN) and/or N-hydroxysuccinimide and/or riboflavin-5-phosphate and/or cupferron and/or tributyl phosphate and/or glutathione and/or black phosphorus and/or graphene and/or mercury and/or bismuth.

### Measurement Techniques

Measurement of diameter and height of nanoclusters 120 was performed using the Nanosurf Easyscan 2 flex AFM (Easy Scan 2 Flex, NanoSurf, Liestal, Switzerland) to characterize the pattern of the clusters before and after the electrochemistry measurements. The AFM analyses indicated that the clusters bonded firmly to the electrode surface and did not disconnect from it after the electrochemistry measurements. The scans were also analyzed with dedicated Matlab code to determine clusters diameter and height using the data matrix obtained from the AFM device. Two cursors were placed in the cluster's center in the measurement process, one along the x-axis and the other along the y-axis. We used the cursors to measure the diameters of the cluster bases and the shape of the nanoclusters profile in the z-axis cross-section.

In this measurement method, the diameter dimensions were defined between two points. The location of the two selected points was defined at the edge of the nanoclusters with a z value slightly above the average z value of the smooth surface, where a continuous increase in the z values was observed between these selected two points toward the cluster center. Cluster height was defined as the z value in the cluster's center (highest point).

### Exemplary Sensitivities

Patterning lithography methods produce a pattern of nanoclusters by controlling the pitch between them and by forming and controlling the size of the nanoclusters in a simple process. Controlling pitch (see FIGS. 11-16), and nanocluster size (See Table 3 and Table 4 and/or FIGS. 17-18b) contributes to sensitivity of detection and/or to an ability to detect two or more cations in parallel.

Table 3 and FIG. 17 indicate that the cluster size contributes to the sensitivity. Table 4 and FIG. 18 indicate that the same surface area to volume ratio gives the same sensitivity.

The large surface area to volume ratio of the nanoclusters and the pitch contributes to the redox potential. In some embodiments the redox potential contributes to an electrical output signal of the sensor. These features contribute to a decrease in interference(s). In some embodiments a decrease in interference(s) contributes to an increase in detection sensitivity.

Alternatively or additionally, in some embodiments patterning lithography methods contribute to an ability to distribute patterns of nanoclusters with ligands specific to different cations. In some embodiments the flexibility of patterning lithography contributes to customizability of configuration of working electrodes. In some embodiments, a working electrode is prepared according to specific requirements provided by a customer.

In some exemplary embodiments of the invention, the ink composition includes a ligand that causes different redox potential for each cation, FIG. 2, FIG. 5-FIG. 9, therefore producing a different electrical signal for each cation. These different redox potentials contribute to an ability to detect different cations in parallel as a sensor array, FIG. 6-FIG. 9.

The above characteristics produce a detection sensitivity in the ppb-ppt (parts per billion to parts per trillion) range for at least two cations detected in parallel, FIG. 2, FIG. 5-FIG. 18.

### Additional Exemplary Ligands

Table 1 presents a list of additional ligand types potentially useful in the preparation of additional exemplary embodiments of the invention. Using this specification as a guide, a person of ordinary skill in the art will be able to implement these ligands in working electrodes, production methods and inks as described hereinabove.

TABLE 1
heavy metals and ligands for their detection

| Heavy metal | Ligand group | Example |
|---|---|---|
| Cadmium | Amines | o-phenylenediamine |
| | Amines/Carboxylates | DTPA-diethylenetriaminepentaacetate |
| | Macrocycle/Crown ether/ | Cal [symbol] x [symbol] 4 [symbol] arene |
| | Calixarenes | |
| | Thi [symbol] s | Thiomalic acid |
| | Pyridines | Pyridylpyrazole-β-ketoenol |
| | Aminopolycarboxylic acid | Nitrilo triacetic acid(NTA) |
| Chromium | Phosphonate compounds | Nitril [symbol] tris(methylene) [symbol] tr [symbol] phosphonic acid [symbol] ATMP [symbol] |
| | Amino acid | Glycine |
| | Carboxylates | Oxalic acid |
| Lead | Thiols | 2,3-Dimercaptopropanol, meso-2,3-Dimercaptosuccinic acid |
| | Amines-carboxylates | Edetate calcium disodium |
| | Amines | Penicilamine |
| | Triazene | 1-(2-ethoxyphenyl)-3-(4-nitrophenyl)tri [symbol] ene (ENT) |
| | Macrocycle/Crown ether/ | Calix [symbol] 4 [symbol] arene |
| | Calixarenes | |
| | Aminopolycarboxylic acid | Nitril [symbol] triacetic acid(NTA) |
| Mercury | Thiols | 2,3-Dimercaptopropan [symbol] l, |
| | | meso-2,3-Dimercaptosuccinic acid, |
| | | 2,3-Dimercaptopropane-1-sulfonic acid |
| | Macrocycle/Crown ether/ | Calix [symbol] 4 [symbol] arene |
| | Calixarenes | |
| | Aminopolycarboxylic acid | Nitrilo triacetic acid(NTA) |
| C [symbol] per | Carboxylates | Dipicolinic acid (DPA), [symbol] -diaminopimelic acid |
| | Thio [symbol] s | Thiomalic acid |
| | Peptide | Glutathione |
| Nickel | Macrocycle/Crown ether/ | Calix [symbol] 4 [symbol] arene |
| | Calixarenes | |
| | Carboxylic acid | Dipicolinic acid (DPA) |
| Uranium | phosphonate compounds | Nitrilotris(methylene) [symbol] tris [symbol] phosphonic acid (ATMP) [symbol] |
| | | Tributyl phosphate |
| | Macrocycle/Crown ether/ | Calix [symbol] 4 [symbol] arene |
| | Calixarenes | |
| | Carboxylates | Salicylic acid |

[symbol]

indicates data missing or illegible when filed

In some exemplary embodiments of the invention, a single ligand binds two or more metals, FIG. 2 and FIG. 5 NTPH for uranyl and lead, FIG. 6 Glu for lead and mercury, FIG. 7 DPA for lead and copper, FIG. 8 DAN for lead and cadmium.

It is expected that during the life of this patent many new ligands specific for heavy metals will be developed and the scope of the invention includes all such new technologies a priori.

Although the invention has been described in conjunction with specific embodiments thereof, it is evident that many alternatives, modifications and variations will be apparent to those skilled in the art. Accordingly, it embraces all such alternatives, modifications and variations that fall within the broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and do not limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of dip pen nanolithography but might also be used in the context of other printing methods such as soft lithography and nanoimprint lithography.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Heavy Metals Nanosensor Development

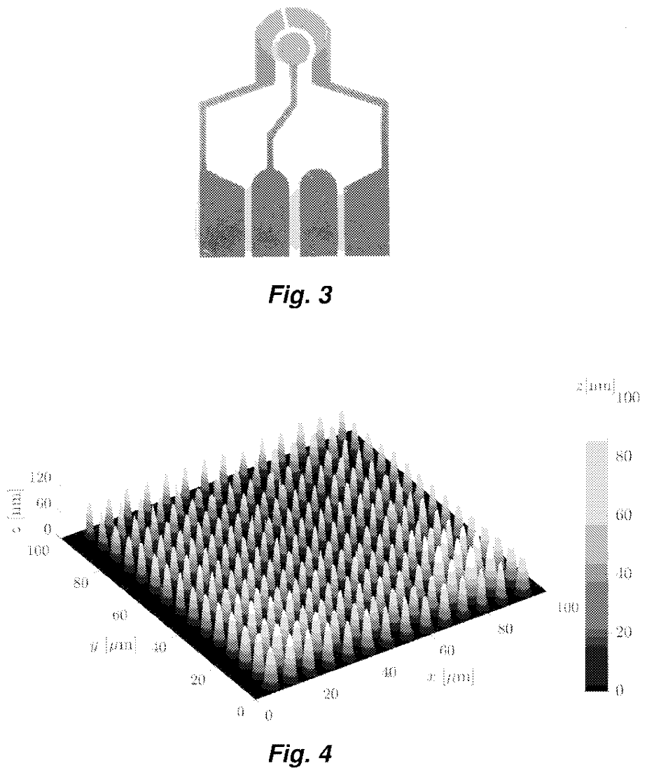

In order to establish/develop a nanosensor array to detect a few metals in parallel in the same electrochemistry measurement the following experiment was done:
1. Ink Preparation, Table 2 summarizes examples of the main kinds of ink composition and ligands.
2. Patterning procedure: in the patterning procedure, less than 1 μL of ink was patterned as clusters on the working electrode surface. The clusters were patterned at different pattern pitches (4 μm, 6.6 μm, 10 μm); the pattern pitch definition is described in FIG. 1. The patterning process was done by NLP2000 System (the instrument is based on dip pen nanolithography, DPN technique) on the working electrode surface of a commercial Thin-film Single MICRUX™ electrode. FIG. 3 and FIG. 4 describe an AuAuAu MICRUX™ electrode and a scanned surface of a pattered MICRUX™ electrode, respectively.
   Electrode naming convention—The name of the electrode indicates the kind of Thin-film Single MICRUX™ electrode (AuAuAu or PtPtPt or PtAuPt), the solvent (a type of polymer, for example, PMMA), the ligand, the pattern pitch. For example: if the MICRUX™ electrode is made from gold-AuAuAu (the working, reference, and counter electrodes made by Au), the solvent is PMMA, the ligand is L-Glutathione, and the pattern pitch is 6.6 μm—the electrode name is AuAuAuPMMAGlu6.6.
3. The pattern was scanned by an AFM and analyzed by dedicated Matlab code, FIG. 4.
4. Electrochemical measurements of samples include heavy metals or metals with different pH and ionic strengths. The samples differ by the concentrations of the heavy metals.
5. 5 μl of each sample solution was dropped on the working electrode surface.
6. Control experiments were done (See FIG. 9 and FIG. 10): FIG. 9 (graph Buffer pH 4.6) a result of a solution without metals (only buffer acetate pH 4.6), which was measured by Micrux electrode AuAuAuPMMAHg6.6 (a patterned electrode). FIG. 10. (graphs AuAuAu and AuAuAuPMMA) graphs AuAuAu and AuAuAuPMMA are measurements of solutions that include metals that were measured by electrodes that were not patterned (AuAuAu) and which were patterned only with PMMA without a ligand (AuAuAuPMMA). The results indicate that without a ligand there are no waves which are the electrical signals of the metals in the solutions.
   Results are presented in tables 3-4 and FIGS. 2 and 4 to 18b.
   The results illustrate that:
1. The nanosensor can detect metals in the ppt-ppb concentrations range (FIGS. 2, 5-18b).
2. The nanosensor can detect two or more metals in parallel (FIGS. 6-9, 18a and 18b). Control experiments indicate that pattern electrodes did not detect metals if the solutions did not include metals, FIG. 9 graph Buffer pH 4.6 (i.e. no falso positives). Electrodes that were not patterned AuAuAu, or electrodes patterned only with PMMA, AuAuAuPMMA, did not detect heavy metals when the solutions include metals, FIG. 10 (graphs AuAuAu and AuAuAuPMMA).
3. The pattern pitch influences the redox potential (FIGS. 11-16).
4. The cluster size, especially the surface area to volume ratio, affects the sensitivity. (Tables 3 and 4, FIG. 17 and FIGS. 18a and 18b).
5. One ligand can detect/entrap/bind to more than one metal and give different electrical signals, FIG. 6 (L=Glu), FIG. 7 (L=DPA), FIG. 8 (L=DAN), FIG. 9 (L=Hg)

TABLE 2
Ink composition

| | PMMA | Acetonitrile | mass Ligand | |
|---|---|---|---|---|
| Ink name | (%V) | (%V) | (mg) | Ligand structure |
| PMMA-Glu PMMA-PolyMethyl methacrylate Glu-L-Glutathione | 50% | 50% | 30.0 | 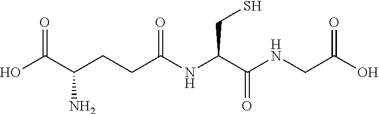 [SMILES: N[C@@H](CCC(=O)N[C@@H](CS)C(=O)NCC(=O)O)C(=O)O] |
| PMMA-DPA PMMA-PolyMethyl methacrylate DPA-Pyridine-2,6-dicarboxylic acid | 50% | 50% | 16.7 | 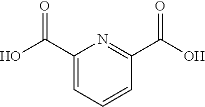 [SMILES: O=C(O)c1cccc(C(=O)O)n1] |
| PMMA-PA PMMA-PolyMethyl methacrylate PA-Penicillamine | 50% | 50% | 14.9 | 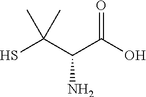 |
| PMMA-DAN PMMA-PolyMethyl methacrylate DAN-1,8-Diaminonaphtalene | 50% | 50% | 15.8 | 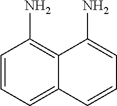 [SMILES: Nc1cccc2cccc(N)c12] |
| PMMA-NTPH PMMA-PolyMethyl methacrylate NTPH- nitrilotris(methylene)]tris(phosphonic acid) | 50% | 50% | 10.0 | 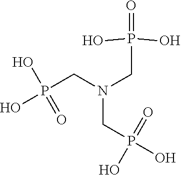 [SMILES: O=P(O)(O)CN(CP(=O)(O)O)CP(=O)(O)O] |
| PMMA-Hg PMMA-PolyMethyl methacrylate Hg | 50% | 50% | 10.0 | Mercury acetate |

TABLE 3
The effect of the surface area to volume ratio on the sensitivity (AuAuAuPMMANTPH6.6 electrodes)

| | AuAuAuPMMANTPH-1 | AuAuAuPMMANTPH-2 | AuAuAuPMMANTPH-3 | AuAuAuPMMANTPH-4 |
|---|---|---|---|---|
| Average height | 14 | 16 | 20 | 148 |
| [nanometers] | | | | |
| major axis | 1.94 | 1.89 | 2.39 | 3.74 |
| average | | | | |
| [micrometers] | | | | |
| minor axis | 1.66 | 1.68 | 1.94 | 0.88 |
| average | | | | |
| [micrometers] | | | | |
| Surface area to | 189 | 190 | 60 | 63 |
| volume ratio | | | | |
| [micrometers [symbol] ] | | | | |

[symbol]

indicates data missing or illegible when filed

TABLE 4
The effect of the surface area to volume ratio
on the sensitivity (AuAuAuPMMAGlu electrodes)

| | AuAuAuPMMAGlu-1 | AuAuAuPMMAGlu-2 |
|---|---|---|
| Average height | 142 | 144 |
| [nanometers] | | |
| Major axis average, | 4.16 | 4.19 |
| diameter [micrometers] | | |
| Minor axis average, | 0.88 | 0.96 |
| diameter [micrometers] | | |
| Surface area to volume | 44 | 49 |
| ratio [micrometers⁻¹] | | |

FIG. 3 is a schematic representation of a Thin-film Gold MICRUX™ single electrode (AuAuAu) suitable for use in some embodiments of the invention.

FIG. 4 is a heat map of thickness produced by Matlab analysis of AuAuAuPMMANTPH pattern scanning.

FIG. 5 illustrates Square wave anodic stripping voltammetry of MICRUX™ electrode AuAuAuPMMANTPH6.6 (E deposition=−1.1 (V); t deposition=60 s; Frequency=25 Hz; Amplitude=0.1V). The solution includes Pb(NO₂)₂, 1.0×10⁻⁶ M KNO₃, PH 5.5.

FIG. 6 illustrates Square wave anodic stripping voltammetry of MICRUX™ electrode AuAuAuPMMAGlu6.6 (E deposition=−1.1 (V); t deposition=60 s; Frequency=25 Hz; Amplitude=0.1V). The solution includes Pb(NO₂)₂ and Hg(NO₂)₂, 1.0×10⁻⁶ M KNO₃, PH 5.5.

FIG. 7 illustrates Square wave anodic stripping voltammetry of MICRUX™ electrode AuAuAuPMMADPA6.6 (E deposition=−1.1 (V); t deposition=60 s; Frequency=25 Hz; Amplitude=0.075V). The solutions include Pb(NO₂)₂ and Cu(NO₂)₂, ionic strength 5.0×10⁵ M, pH 3.0.

FIG. 8 illustrates Square wave anodic stripping voltammetry of MICRUX™ electrode AuAuAuPMMADAN6.6 (E deposition=−1.2 (V); t deposition=60 s; Frequency=25 Hz; Amplitude=0.01V). The solutions include Pb(NO₂)₂ and Cd(NO₂)₂, ionic strength 5.0×10⁻⁵ M, pH 3.0.

FIG. 9 illustrates Square wave anodic stripping voltammetry of MICRUX™ electrode AuAuAuPMMAHg6.6 (E deposition=−1.2 (V); t deposition=60 s; Frequency=25 Hz; Amplitude=0.1V). The solutions include Cd(NO₂)₂ Pb(NO₂)₂ and Cu(NO₂)₂, ionic strength 5.0×10⁻⁵ M, buffer acetate pH 4.6. Gray line—The solutions include ionic strength 5.0×10⁻⁵ M, buffer acetate pH 4.6.

FIG. 10 illustrates Square wave anodic stripping voltammetry of MICRUX™ electrode (E deposition=−1.2 (V); t deposition=60 s; Frequency=25 Hz; Amplitude=0, 1V). The solutions include ionic strength 5.0×10⁻⁵ M, Buffer acetate pH 4.6. Gray line-AuAuAuPMMAHg6.6 electrode, 0.10 ppb Cd(NO₂)₂ Pb(NO₂)₂ and Cu(NO₂)₂, Black line—AuAuAu electrode, Buffer acetate pH 4.6. Broken black line-AuAuAu electrode, 1.0 ppb Cd(NO₂)₂ Pb(NO₂)₂ and Cu(NO₂)₂, Dotted black line AuAuAuPMMA electrode, 1.0 ppb Cd(NO₂)₂ Pb(NO₂)₂ and Cu(NO₂)₂.

FIG. 11 is a calibration curve of Hg(NO₂)₂—MICRUX™ electrode AuAuAuPMMAGlu (E deposition=−1.1 (V); t deposition=60 s; Frequency=25 Hz; Amplitude=0.1V). The solution includes Buffer acetate pH 4.6. ionic strength 5.0×10⁻⁵ M.

FIG. 12 illustrates Square wave anodic stripping voltammetry of MICRUX™ electrode AuAuAuPMMAGlu (E deposition=−1.1 (V); t deposition=60 s; Frequency=25 Hz; Amplitude=0.1V). The solution includes 0.50 ppb Hg(NO₂)₂, Buffer acetate pH 4.6. ionic strength 5.0×10⁻⁵ M.

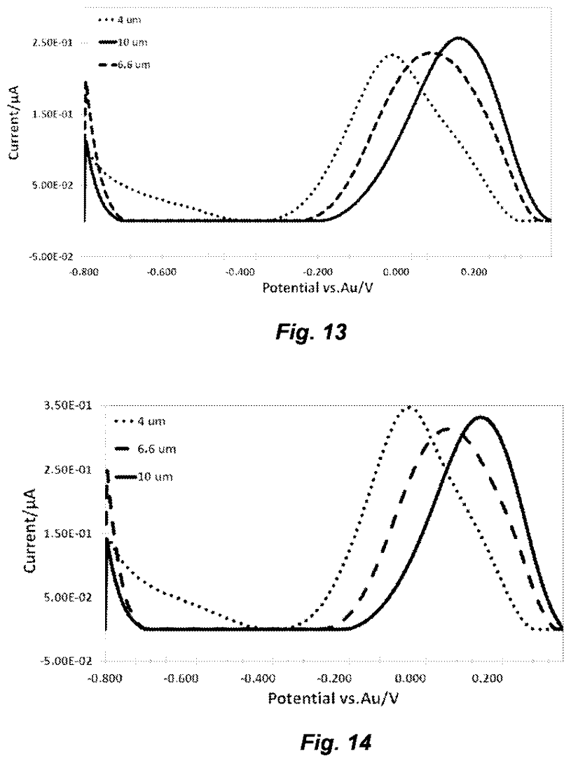

FIG. 13 illustrates Square wave anodic stripping voltammetry of MICRUX™ electrode AuAuAuPMMAGlu (E deposition=−1.1 (V); t deposition=60 s; Frequency=25 Hz; Amplitude=0.1V). The solution includes 1.0 ppb Hg(NO₂)₂, Buffer acetate pH 4.6. ionic strength 5.0×10⁻⁵ M.

FIG. 14 illustrates Square wave anodic stripping voltammetry of MICRUX™ electrode AuAuAuPMMAGlu (E deposition=−1.1 (V); t deposition=60 s; Frequency=25 Hz; Amplitude=0.1V). The solution includes 2.0 ppb Hg(NO₂)₂, Buffer acetate pH 4.6. ionic strength 5.0×10⁻⁵ M.

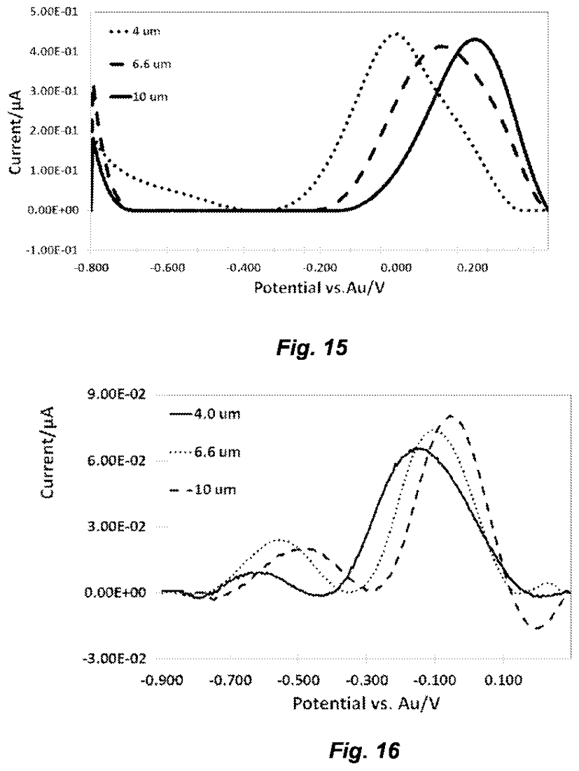

FIG. 15 illustrates Square wave anodic stripping voltammetry of MICRUX™ electrode AuAuAuPMMAGlu (E deposition=−1.1 (V); t deposition=60 s; Frequency=25 Hz; Amplitude=0.1V). The solution includes 4.0 ppb Hg(NO₂)₂, Buffer acetate pH 4.6. ionic strength 5.0×10⁻⁵ M.

FIG. 16 illustrates Square wave anodic stripping voltammetry of MICRUX™ electrode AuAuAuPMMAGlu (E deposition=−1.1 (V); t deposition=60 s; Frequency=25 Hz; Amplitude=0.1V). The solution includes 0.50 ppb Hg(NO₂)₂ and 0.50 ppb Pb(NO₂)₂, Buffer acetate pH 4.6, ionic strength 5.0×10⁻⁵ M.

FIG. 17 is a calibration curve of MICRUX™ electrodes AuAuAuPMMANTPH6.6 (E deposition=−1.1 (V); t deposition=60 s; Frequency=25 Hz; Amplitude=0.1V). The solution includes buffer acetate pH 4.6; ionic strength 5.0×10⁻⁵ M, Pb(NO₂)₂.

FIG. 18a is a calibration curve of MICRUX™ electrode AuAuAuPMMAGlu6.6 (E deposition=−1.1(V); t deposition=60 s; Frequency=25 Hz; Amplitude=0.1V). The solution includes buffer acetate pH 4.6. ionic strength 5.0×10⁵ M Hg(NO₂)₂.

FIG. 18b is a calibration curve of MICRUX™ electrode AuAuAuPMMAGlu6.6 (E deposition=−1.1(V); t deposition=60 s; Frequency=25 Hz; Amplitude=0.1V). The solution includes buffer acetate pH 4.6. ionic strength 5.0×10⁵ M Pb(NO₂)₂.

What is claimed is:

1. A working electrode comprising:
(a) a conductive surface; and
(b) a plurality of nanoclusters of one or more ligands specific to one or more metals distributed on said conductive surface with a controlled pitch.

2. A working electrode according to claim 1, wherein said nanoclusters have a height of at least 2 nm.

3. A working electrode according to claim 1, wherein said nanoclusters have a height of 1000 nm or less.

4. A working electrode according to claim 1, wherein said nanoclusters have a diameter of at least 10 nm.

5. A working electrode according to claim 1, wherein said nanoclusters have a diameter of 6000 nm or less.

6. A working electrode according to claim 1, wherein said controlled pitch is at least 10 nm.

7. A working electrode according to claim 1, wherein said controlled pitch is 11,000 nm or less.

8-10. (canceled)

11. A working electrode according to claim 1, configured to detect at least two metals concurrently in a single sample.

12. A working electrode according to claim 11, wherein said at least two metals are selected from the group consisting of cadmium, chromium, lead, mercury, nickel, copper, cerium, vanadium, molybdenum, and uranium (in their various oxidation states).

13. A method comprising:
applying one or more solutions or suspensions of ligand(s) specific to one or more metals dissolved/suspended in one or more solvents to a conductive surface using patterning lithography to produce nanoclusters with a controlled pitch.

14. A method according to claim 13, comprising controlling a size of said nanoclusters.

15. A method according to claim 14, wherein said controlling includes insuring said nanoclusters have a height of 2 nm to 1000 nm.

16. A method according to claim 14, wherein said controlling includes insuring said nanoclusters have a diameter 10 nm to 6000 nm.

17. A method according to claim 13, wherein said controlled pitch is 10 nm to 11,000 nm.

18. A method according to claim 13, comprising dissolving said ligand(s) in said one or more solvents at a concentration of 1.00×10⁻⁸ M to 0.75 M.

19. A method according to claim 13, wherein said conductive surface includes at least one member of the group consisting of ITO (indium tin oxide), FTO (fluorine doped tin oxide), Pt (platinum), Au (gold), Glassy carbon, and semiconductor material(s).

20-21. (canceled)

22. A patterning ink comprising:
a ligand with specificity to one or more metals dissolved/suspended in a solvent or mixture of solvents.

23. An ink according to claim 22, comprising at least 1% solvent by volume.

24. An ink according to claim 22, comprising less than 75% solvent by volume.

25. An ink according to claim 22, comprising from 1.00×10⁻⁸ M to 0.75M of said ligand.

26-27. (canceled)
